(19) United States
(12) Patent Application Publication
(10) Pub. No.: US 2026/0210953 A1
(43) Pub. Date: Jul. 23, 2026

(54) A METHOD OF PRODUCING BIOMOLECULE-CROSSLINKED COMPOUND AND A COMPOUND DERIVED THEREOF

(71) Applicant: CHIANG MAI UNIVERSITY, Chiang Mai (TH)

(72) Inventors: Nipon THEERA-UMPON, Chiang Mai (TH); Suruk UDOMSOM, Lamphun (TH); Pathinan PAENGNAKORN, Chiang Mai (TH); Phornsawat BAIPAYWAD, Chiang Mai (TH)

(73) Assignee: CHIANG MAI UNIVERSITY, Chiang Mai (TH)

(21) Appl. No.: 19/144,126

(22) PCT Filed: Feb. 12, 2024
(86) PCT No.: PCT/TH2024/050004

§ 371 Date: Jun. 27, 2025

(30) Foreign Application Priority Data
Feb. 21, 2023 (TH) 2301000943

(51) Int. Cl. G01N 33/543; G01N 33/531; G01N 33/552

(52) U.S. Cl. CPC G01N 33/54346; G01N 33/531; G01N 33/552

(57) ABSTRACT

A method of producing a biomolecule-crosslinked compound is disclosed. More specifically, the method comprises the steps of reacting a plurality of monomers and a plurality of comonomers in the presence of ammonia within a first solution to generate a compound; retrieving the produced compound from the first solution for cleaning; subjecting the cleaned compound to react with a second solution to join a crosslinker onto the compound through a first amine functional group of the crosslinker; and adding a plurality of biomolecules to the second solution to bind the biomolecules towards the compound through a second amine functional group of the crosslinker to produce the biomolecule-crosslinked compound.

### RELATED APPLICATIONS

This application is a National Stage filing under 35 U.S.C. § 371 of International Application No. PCT/TH2024/050004 filed Feb. 12, 2024, which claims priority to Thailand application Ser. No. 23/010,00943 filed Feb. 21, 2023, which applications are incorporated herein by reference.

### TECHNICAL FIELD

The present disclosure relates to a method of producing a compound capable of crosslinking a biomolecule to form a biomolecule-crosslinked compound. More particularly, the bond crosslinking the compound and the biomolecule is realized by interaction with amine functional groups located on a crosslinker. Also, the present disclosure is associated with the biomolecule-crosslinked compound and derivatives derived thereof.

### BACKGROUND

A rapid growth of nanomaterial technology has been contributing significantly to a recent advance in biosensor development.¹ Nanoparticles (NPs) are defined as small particles and they usually exhibit distinct properties due to their high surface-to-volume ratio which are useful for biosensing. In principle, a biosensor relies on a specific interaction between an analyte and a bio-recognition element on the biosensor, such as substrate-enzyme or antigen-antibody.^{2, 3} Therefore, immobilization of biomolecules on nanoparticles is a promising strategy to enhance the sensitivity and selectivity of the biosensor.^{4, 5} Various nanoparticles functionalized with biomolecules have been established and applied on biosensing applications, such as carbon nanotubes, gold nanoparticles, and other metallic nanoparticles.⁶⁻⁸ For example, glutaraldehyde is a crosslink for coating a great amount of bioreceptors on the surface of nanoparticles. It can link to many kinds of bioreceptors (i.e. proteins, genetic materials) but in an undesirable non-specific manner. U.S. Pat. No. 7,226,794B2 disclosed gold nanoparticles (AuNPs) capable of using surface enhanced Ramen spectroscopy (SERS) for determining presence of biomolecules. Further European patent application no. 1794590 A4 disclosed method of using single wall carbon nanotube for immobilizing biomolecules on the carbon nanotube.

Further, silica nanoparticles (SNPs) are one of the inorganic nanomaterials that have been of interest for biomolecule immobilization. SNPs possess desirable characteristics such as biocompatibility, mechanical stability, and tunable size and shape.⁹ SNPs could be formed in various shapes and structures resulting in different properties, for example, conventional nonporous SNPs, ¹⁰ mesoporous silica nanoparticles, ^{11, 12} hollow mesoporous silica nanoparticles ¹³, and core-shell silica. ¹⁴ Due to their versatile properties, SNPs have been exploited in a wide range of biomedical applications such as drug delivery, therapeutics and diagnostics.^{9, 15, 16} It has been previously reported that amine-functionalized SNPs are biocompatible and suitable for use as a drug delivery system. 3-aminopropyl)triethoxysilane (APTES) has been used to provide an amine group for generating amine-functionalized silica nanoparticles (AFSNPs). In the conventional method, APTES was coated on the surface of the SNP via condensation reaction.¹⁸⁻²¹ However, there were some limitations, such as the uncontrollable number of functional groups, limited shelf-life stability, and random orientation of the molecules, leading to reduce effectiveness of biomolecule immobilization. It could ultimately decrease the sensitivity in biosensing applications.

There are several strategies employed for immobilization^{22, 23}; however, they can be classified into two main categories: non-covalent and covalent attachment. Non-covalent bonding relies on electrostatic, hydrophobic, polar, and affinity interactions such as biotin-avidin bonding and histidine-metal chelation. Meanwhile, covalent bonding involves the formation of bonds between the functional groups of biomolecules and the surface of materials, such as amine, carboxyl, thiol, aldehyde, or alkyne²⁴. To immobilize a biomolecule on the SNPs, surface functionalization of the SNPs is required before the addition of a crosslink between the SNPs and biomolecules. The most commonly used crosslinkers for biomolecules immobilization are glutaraldehyde and 1-ethyl-3-(3-dimethylaminopropyl)-carbodiimide/N-hydroxysuccinimide (EDC/NHS)²⁵. However, the limitation of these crosslinkers is that they can only crosslink between the N-terminal and C-terminal. Therefore, there exist a need to enable biomolecule immobilization onto the surface of the nanoparticle through interaction of functionalized groups other than cross-linkage between N-terminal and C-terminal only to widen potential application of the biosensor developed based on the like principle.

### SUMMARY

The present disclosure is directed to a method for producing a compound capable of crosslinking a biomolecule or a biomolecule-crosslinked compound. More specifically, the disclosed method uses a pair of compatible monomers and comonomers to yield SNPs or the compounds in a single step reaction, which is much simplified compared to conventional approach to synthesize the compounds.

Further object of the present disclosure is to offer a method of producing the SNPs or SNP-based compounds under a relatively mild condition with a temperature around or slightly above of room temperature.

More object of the present disclosure is to cater a method of producing biomolecule-crosslinked compounds or biomolecule-crosslinked SNPs in a controlled manner by using a designated and modified crosslinker enabling crosslinking of the biomolecules, modified or not, and the compounds preferably through functionalized amine groups instead of conventional N-terminal and C-terminal interaction.

Still, another object of the present disclosure is directed to a biomolecule-crosslinked compound, in which the biomolecule is crosslinked towards the compound through a crosslinker possessing a pair of amine functional groups capable of binding both the compound and the biomolecule.

At least one of the preceding objects is met, in whole or in part, by the present disclosure, in which one of the embodiments of the present disclosure is a method of producing a biomolecule-crosslinked compound. The method essentially comprises the steps of reacting a plurality of monomers and a plurality of comonomers in the presence of ammonia within a first solution to generate a compound; retrieving the produced compound from the first solution for cleaning; subjecting the cleaned compound to react with a second solution to join a crosslinker onto the compound through a first amine functional group of the crosslinker; and adding a plurality of biomolecules to the second solution to bind the biomolecules towards the compound through a second amine functional group of the crosslinker to produce the biomolecule-crosslinked compound. Preferably, the monomers are Tetrapropyl orthosilicate (TPOS), Tetraethyl orthosilicate (TEOS), Tetramethyl orthosilicate (TMOS), Tetra-isopropyl orthosilicate or Butyl orthosilicate. Preferably, the comonomers are selected from (3-Aminopropyl)-triethoxysilane), (3-Aminopropyl)-trimethoxysilane), (3-Aminopropyl-methyl-diethoxysilane), (3-(Dimethoxymethylsilyl) propylamine), (3-Aminopropylsilanetriol), (N-(2-Aminoethyl) (3-aminopropyl)methyldimethoxysilane) or N-(2-Aminoethyl) (3-aminopropyl) trimethoxysilane).

According to some embodiments, the disclosed method further comprises precipitating the produced biomolecule-crosslinked compound and dispersing the precipitated biomolecule-crosslinked compound in a distilled water.

According to more embodiments, the monomers and the comonomers are in a molar ratio of 1.3-4.0:0.4-3.0. More preferably, in other embodiments, the first solution comprises ammonium hydroxide dissolved in a mixture of distilled water and ethanol in a volume ratio of 1-10:20-30.

According to several embodiments, the second solution comprises itaconic acid, 1-Ethyl-3-(3-dimethyl aminopropyl) carbodiimide (EDC) and N-hydroxy succinimide (NHS) dispersed in an aqueous phase at a molar ratio of 15-35:60-90:30-60.

According to several embodiments, the reacting step is performed at a temperature range of 20° C. to 40° C.

According to some embodiments, the biomolecule is any of antigen, antibody, peptide, DNA or RNA incorporated with an amino-terminus capable of binding towards the second amine functional group of the crosslinker.

Another aspect of the present disclosure biomolecule-crosslinked compound of a chemical structure:

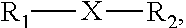

wherein X is a crosslinker or a formula of
(i) —HN—C(═O)—CH₂C(═CH₂)—C(═O)—NH— or
(ii) —HN—C(═O)—CH₂C(═CH₂)—C—NH—. Preferably, R₁ is a silica nanoparticle and R₂ is a biomolecule incorporated with an amino-terminus capable of binding towards an amine functional group of the crosslinker.

For a number of embodiments of the disclosed compound, the crosslinker is produced by reacting itaconic acid, 1-Ethyl-3-(3-dimethyl aminopropyl) carbodiimide (EDC) and N-hydroxy succinimide (NHS) dispersed in an aqueous phase at a molar ratio of 15-35:60-90:30-60.

For several embodiments of the disclosed compound, the silica nanoparticle comprises monomers and comonomers prepared in a molar ratio of 1.3-4.0:0.4-3.0. Preferably, the monomers are Tetrapropyl orthosilicate (TPOS), Tetraethyl orthosilicate (TEOS), Tetramethyl orthosilicate (TMOS), Tetra-isopropyl orthosilicate or Butyl orthosilicate. Preferably, the comonomers are selected from (3-Aminopropyl)-triethoxysilane), (3-Aminopropyl)-trimethoxysilane), (3-Aminopropyl-methyl-diethoxysilane), (3-(Dimethoxymethylsilyl) propylamine), (3-Aminopropylsilanetriol), (N-(2-Aminoethyl) (3-aminopropyl)methyldimethoxysilane) or N-(2-Aminoethyl) (3-aminopropyl) trimethoxysilane).

Further aspect of the present disclosure pertains to a method of producing amine-functionalized silica nanoparticles (AFSNPs) comprising reacting a plurality of monomers and a plurality of comonomers in the presence of ammonia within a first solution to generate the AFSNPs; and retrieving the produced AFSNPs from the first solution for cleaning. Preferably, the monomer is Tetrapropyl orthosilicate (TPOS), Tetraethyl orthosilicate (TEOS), Tetramethyl orthosilicate (TMOS), Tetra-isopropyl orthosilicate or Butyl orthosilicate, and the comonomer is selected from (3-Aminopropyl)-triethoxysilane), (3-Aminopropyl)-trimethoxysilane), (3-Aminopropyl-methyl-diethoxysilane), (3-(Dimethoxymethylsilyl) propylamine), (3-Aminopropylsilanetriol), (N-(2-Aminoethyl) (3-aminopropyl)methyldimethoxysilane) or N-(2-Aminoethyl) (3-aminopropyl) trimethoxysilane).

In more embodiments of the method producing the AFSNPs, the steps further comprise subjecting the cleaned AFSNPs to react with a second solution to join a crosslinker onto the compound through a first amine functional group of the crosslinker. Preferably, the crosslinker is of a formula of:

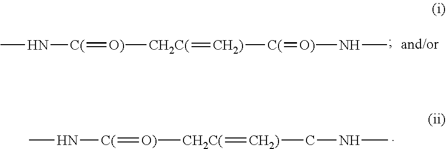

### BRIEF DESCRIPTION OF THE DRAWINGS

FIG. 1 shows schematic of the synthesis of amine-functionalized silica nanoparticles (AFSNPs) through (A) a one-step synthesis of the disclosed method and (B) conventional synthesis;

FIG. 2 shows schematic of the immobilization of biomolecules on ASFNPs surface by using itaconic acid as the crosslinker;

FIGS. 3 (A) and (B) shows morphologies of silica NPs and AFSNPs while (C) and (D) shows size distribution of silica NPs and AFSNPs;

FIG. 4 shows SEM images of the AFSNPs with varying of APTES concentrations (A) 30 mol %, (B) 50 mol %, (C) 70 mol %, and (D) ζ-potential measurement of AFSNPs;

FIG. 5 is a graph showing efficiency of APTES concentration in affecting binding of antibodies onto the surface of nanoparticles through ELISA technique;

FIG. 6 is a graph comparing biomolecules immobilization stability between AFSNPs derived from conventional and one-step synthesis of AFSNPs

FIG. 7 shows (A) a schematic illustration of the roles of AFSNPs in improving the performance of conventional ELISA, (B) results of the ELISA tests, and (C) a graph revealing sensing performance of the ELISA test in the presence and absence of AFSNPs;

FIG. 8 is a graph showing specificity of the performed ELISA test for anti-rabies virus detection with the concentration of anti-rabies and BSA was 2.5 IU/mL and 1 mg/mL, respectively.

### DETAILED DESCRIPTION

Hereinafter, the disclosure shall be described according to the preferred embodiments and by referring to the accompanying description and drawings. However, it is to be understood that referring the description to the preferred embodiments of the disclosure and to the drawings is merely to facilitate discussion of the various disclosed embodiments and it is envisioned that those skilled in the art may devise various modifications without departing from the scope of the appended claim.

As used herein, the phrase “in embodiments” means in some embodiments but not necessarily in all embodiments.

As used herein, the terms “approximately” or “about”, in the context of concentrations of components, conditions, other measurement values, etc., means +/−5% of the stated value, or +/−4% of the stated value, or +/−3% of the stated value, or +/−2% of the stated value, or +/−1% of the stated value, or +/−0.5% of the stated value, or +/−0% of the stated value.

The terms “amine-functionalized silica nanoparticles” (AFSNPs) and “conjugate” are used interchangeably throughout the present disclosure referring to a substance synthesized using the preferred monomers and comonomers according to methods and conditions at a ratio or proportion described hereinafter with a functional amino-terminus capable of binding towards an amine functional group located at a crosslinker joining the substance to a biomolecule through a covalent bonding.

The term “biomolecule” used herein throughout of the specification shall refer to one or more chemical compounds generally derived from living organisms or the like such as virus. These biomolecules may be subjected to any known pretreatments and/or modifications prior to being joined to the AFSNPs to attain at least one of the objects mentioned above.

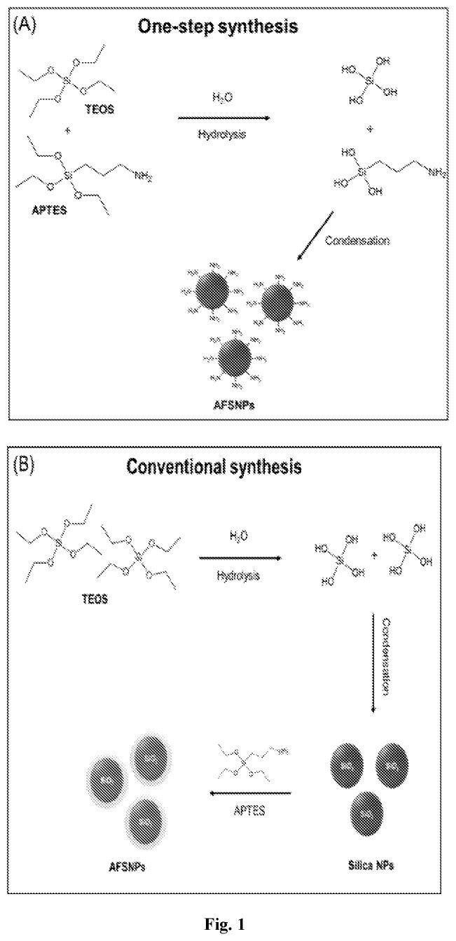

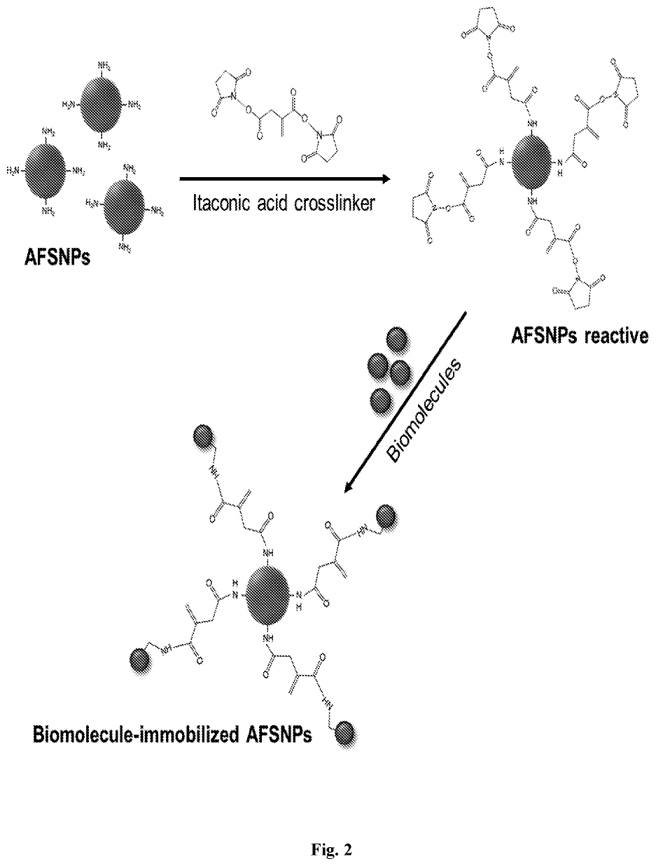

Referring to FIGS. 1A and 2, a method of producing a biomolecule-crosslinked compound is illustrated. Essentially, the method comprises the steps of reacting a plurality of monomers and a plurality of comonomers in the presence of ammonia of a first solution to generate a compound; retrieving the produced compound from the first solution for cleaning; subjecting the cleaned compound to react with a second solution to join a crosslinker onto the compound through a first amine functional group of the crosslinker; and adding a plurality of biomolecules to the second solution to bind the biomolecules towards the compound through a second amine functional group of the crosslinker to produce the biomolecule-crosslinked compound.

According to several embodiments of the disclosed method, the first solution comprises ammonium hydroxide dissolved in a mixture of distilled water and ethanol in a volume ratio of 1-10:20-30. It is important to note that other aqueous soluble ammonium salts such as ammonium carbonate, ammonium chloride, and ammonium nitrate can be employed in some embodiments of the disclosed method for preparation of the first solution instead of using ammonium hydroxide. Likewise, other aqueous miscible alcohol such as n-propyl alcohol, isopropyl alcohol, t-butyl alcohol, etc. can be utilized as well for preparation of the first solution as long the ammonium salts become dissolved and ionized in the first solution enabling the concurrence hydrolysis and condensation reactions of the monomers and comonomers.

For more embodiments, the monomer is Tetrapropyl orthosilicate (TPOS), Tetraethyl orthosilicate (TEOS), Tetramethyl orthosilicate (TMOS), Tetra-isopropyl orthosilicate or Butyl orthosilicate. Correspondingly, the comonomers applicable in the disclosed method to react with the mentioned monomers is selected from (3-Aminopropyl)-triethoxysilane), (3-Aminopropyl)-trimethoxysilane), (3-Aminopropyl-methyl-diethoxysilane), (3-(Dimethoxymethylsilyl) propylamine), (3-Aminopropylsilanetriol), (N-(2-Aminoethyl) (3-aminopropyl)methyldimethoxysilane) or N-(2-Aminoethyl) (3-aminopropyl) trimethoxysilane). The molar ratio of the monomers and comonomers in the first solution is 1.3-4.0:0.4-3.0.

It was found by the inventors of the present disclosure that the disclosed method is preferably conducted under relatively mild condition such that the reactions become controlled and regulated substantially free from producing undesirable side products as found in the conventional approach. Preferably, the reacting step is performed at the room temperature. Still, it is feasible to have the reacting step run at a temperature slightly lower or above of the room temperature. The temperature for the reacting step can be 20 to 45° C. Preferably, the concurring hydrolysis and condensation reaction in the first solution can be expedited by stirring the reactants in a gentle manner for a period of time, preferably 2 to 24 hours depending on the amount of the reactants involved in the reacting step.

After completion of the reacting step, the compounds generated or produced may be recovered from the first solution for cleaning purposes to remove unreacted reagents from the surface of the compounds prior to subjecting the compounds for reaction with the second solution and reagents dispersed within the second solution. The produced compounds can be recovered by way of filtration, centrifugation or any other approaches known in the field. The cleaning step or process avoid cross-contamination of the second solution by the reagents from the first solution. It is possible to keep the clean compounds in a freezer or lyophilized form for a period of time until the clean compounds are brought into contact with the second solution and the reagents dispersed inside the second solution.

Pursuant to a number of the preferred embodiments of the disclosed method, the second solution comprises itaconic acid, 1-Ethyl-3-(3-dimethyl aminopropyl) carbodiimide (EDC) and N-hydroxy succinimide (NHS) dispersed in an aqueous phase at a molar ratio of 15-35:60-90:30-60. It is crucial to note that the itaconic acid is the crosslinker to join the biomolecule to the compounds or AFSNPs. More preferably, the itaconic acid comprises an amine functional group at each of its end that one of the amine functional group serves to conjugate with the AFSNPs or compound and another amine functional group is designated to bind onto the biomolecule. Still, in some embodiments, the itaconic acid may be modified to produce biomolecule-crosslinked compounds with the ideal characteristics. In a number of embodiments of the disclosed methods, the itaconic acids in the second solution may be subjected to react with the compounds and the biomolecules simultaneously such that both functionalized ends of the itaconic acid will have equal opportunity to bind towards either one of the biomolecules or the compounds to finally form the biomolecule-crosslinked compounds. The subjecting and adding steps of the disclosed method are performed in a concurrent manner in these embodiments. In general, the compounds, the biomolecules and the itaconic acids are provided with sufficient time, preferably around 15 to 120 minutes and may be varied according to the amount of these reagents involved, to react prior to recovering the formed biomolecule-crosslinked compounds from the second solution. Gentle stirring can be applied initially after adding the compounds and the biomolecules to mix both biomolecules and the compounds evenly in the second solution to attain better equilibrium for the conjugation to be achieved. For some embodiments, the subjecting and adding steps are conducted in a successive fashion that the biomolecules are administered after the compounds have reacted with the itaconic acids for a predetermined duration or vice versa.

In accordance with other embodiments, the disclosed method further comprises precipitating the produced biomolecule-crosslinked compound and dispersing the precipitated biomolecule-crosslinked compound in a distilled water. The precipitating step can be accomplished by way of centrifugation or filtration, while the dispersing steps is directed to cleaning the acquired biomolecule-crosslinked compounds from any contamination of the unreacted reagent remained on the surfaces of the biomolecule. These remaining unreacted reagents may affect efficiency of the produced biomolecule-crosslinked compounds in detecting or determining other substances, preferably presence in a sample, being targeted by the biomolecules linked to the AFSNPs.

For some embodiments, the biomolecule is any of antigen, antibody, peptide, hormones, DNA or RNA incorporated with an amino-terminus capable of binding towards the second amine functional group of the crosslinker. The biomolecule is designated to bind, conjugate, link, or hybridize with one or more analytes such as corresponding antigens, peptides, complementary DNA, complementary RNA, etc possibly present in a sample derived from a subject to obtain a diagnostic outcome or a measurement results.

Another aspect of the present disclosure is associated with a biomolecule-crosslinked compound of a chemical structure: R₁—X—R₂. Accordingly, the X is a crosslinker of a formula of (i) —HN—C(═O)—CH₂C(═CH₂)—C(═O)—NH— or (ii) —HN—C(═O)—CH₂C(═CH₂)—C—NH—; and R₁ is a silica nanoparticle and R₂ is a biomolecule incorporated with an amino-terminus capable of binding towards an amine functional group of the crosslinker. It is important to note that the disclosed biomolecule-crosslinked compounds can be synthesized through, but not limited to, the abovementioned methods.

It was discovered by the inventors of the present disclosure that the crosslinker can be produced by reacting itaconic acid, 1-Ethyl-3-(3-dimethyl aminopropyl) carbodiimide (EDC) and N-hydroxy succinimide (NHS) dispersed in an aqueous phase at a molar ratio of 15-35:60-90:30-60 at a temperature of 20° C. to 40° C. More specifically, the two carboxylic groups at two ends of itaconic acid react with EDC/NHS forming two amide bonds, one with an amine group dedicated to bind the biomolecules, such as proteins and amine-tagged nucleotides, and another formed amine group to be associated with any amine-functionalized substrates, including the AFSNPs. The ability to bind between the N-terminal and N-terminal is beneficial for biosensor applications, especially amine-functionalized silicon-based platforms, for example, silicon photonic chips, silicon nanowires, and fibre optics.

As described in the setting for the, the compound is preferably synthesized from a plurality of proportional monomers and comonomers at a molar ratio of 1.3-4.0:0.4-3.0. More preferably, Tetrapropyl orthosilicate (TPOS), Tetraethyl orthosilicate (TEOS), Tetramethyl orthosilicate (TMOS), Tetra-isopropyl orthosilicate or Butyl orthosilicate can be used to synthesize some embodiments of the disclosed compounds. Correspondingly, the comonomer is selected from (3-Aminopropyl)-triethoxysilane), (3-Aminopropyl)-trimethoxysilane), (3-Aminopropyl-methyl-diethoxysilane), (3-(Dimethoxymethylsilyl) propylamine), (3-Aminopropylsilanetriol), (N-(2-Aminoethyl) (3-aminopropyl)methyldimethoxysilane) or N-(2-Aminoethyl) (3-aminopropyl) trimethoxysilane) for a number embodiments of the disclosed compounds.

In some embodiments of the disclosed compounds, the biomolecule is any of antigen, antibody, peptide, DNA or RNA carrying an amino-terminus capable of binding towards the second amine functional group of the crosslinker. The biomolecule is designated to bind, conjugate, link, or hybridize with one or more analytes such as corresponding antigens, peptides, complementary DNA, complementary RNA, etc possibly present in a sample derived from a subject to obtain a diagnostic outcome or a measurement results.

The following example is intended to further illustrate the disclosure, without any intent for the disclosure to be limited to the specific embodiments described therein.

### Example 1

Amine-functionalized silica nanoparticles (AFSNPs) are fabricated through the one-step and conventional synthesis as illustrated in FIG. 1. In one-step synthesis, the silicon-based monomers of TEOS and APTES were used to produce and modify surface-functionalized nanoparticles (FIG. 1A). The hydrolysis and condensation reactions were induced by adding base catalyst to the mixed solution, whereas the conventional synthesis of AFSNPs began with the fabrication of bare SNPs via modified Stöber method using TEOS as the silica source (FIG. 1B). Amine functionalization was obtained by adding APTES later to cover on surface of SNPs.²¹ Specifically, the uniform-sized AFSNPs were prepared by a one-step synthesis of modified Stöber reaction. Particularly, TEOS and APTES were added to a solvent mixture of ethanol and distilled water in the presence of ammonium hydroxide as a catalyst for hydrolysis and condensation. The mixture was stirred for 6 h at room temperature. The washing process with centrifugation and redispersion was repeated three times. The final products were collected and lyophilized for next use.

The size and surface charge of the AFSNPs in ethanol were determined using a laser particle analyzer system (NANO ZS, Malvern instruments Ltd., UK). A He-Ne laser was used as the light source. The incident wavelength was 633 nm, and the measurement angle was 173°. ζ-potential measurements were performed at 25° C. using highly dilute colloidal dispersions. The morphologies of the AFSNPs were investigated using scanning electron microscopy (SEM, JSM-IT800). To determine the elemental composition of the particles studied during the electron microscopy measurements, an energy-dispersive x-ray spectroscopy (EDS) detector was used together with the SEM. For the measurement in combination with SEM, diluted colloidal dispersion were dropped onto a copper tape on an aluminium substrate (cleaned with ethanol) to produce well-separated particles on the surface. The chemical bond structure of AFSNPs was confirmed by Fourier transform infrared spectroscopy (FTIR). The spectra from 500 to 4000 cm were recorded by an FTIR spectrometer (Thermo Scientific, U.S.A). The samples were scanned in the attenuated total reflection (ATR) mode.

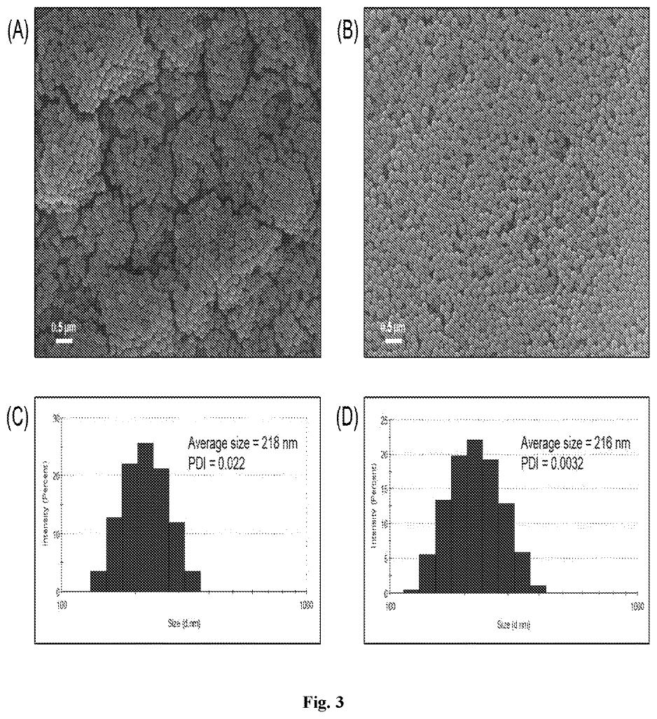

The scanning electron microscope (SEM) images of nanoparticles are shown in FIG. 3. SNPs (FIG. 3A) and AFSNPs (FIG. 3B) displayed the most prominent morphologies in spherical shape with smooth surface and homogeneous diameter. The AFSNPs are slightly smaller in size (diameter around 161±5.9 nm) compared to the bare SNPs (diameter around 164±17.4 nm). Particle size distribution was estimated by the dynamic light scattering (DLS). The histograms of the particle size distribution for the SNPs and AFSNPs are presented in FIGS. 3C and 3D, respectively. The histograms were fitted to the log-normal distribution function and the peak values were taken as average particle size around 218±2.5 nm (SNPs) and 216±1.2 nm (AFSNPs) with a low polydispersity index (PDI) which represents a perfectly uniform particle size and is consistent with the SEM results. However, the different measured particle sizes between SEM and DLS are caused by the heterogeneous populations of the weighting procedure, which are inevitable. ²⁶

### Example 2

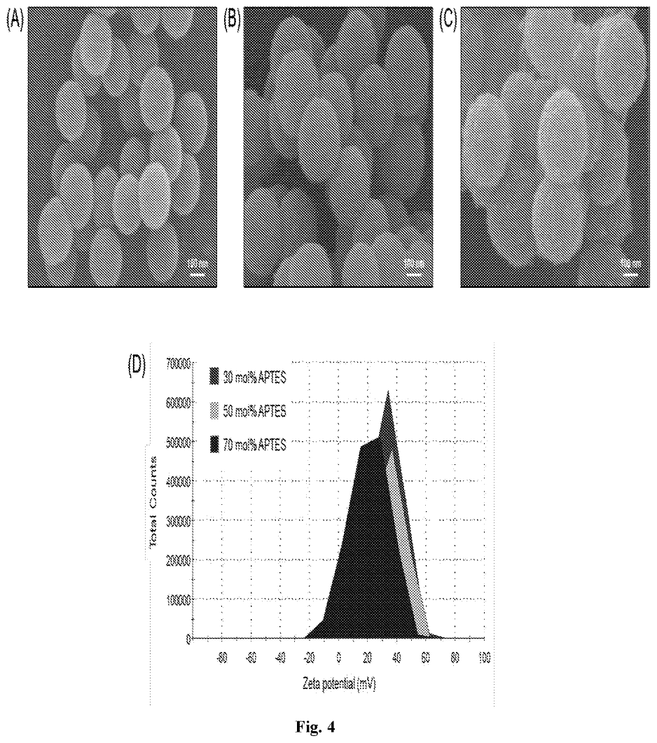

In addition, inventors of the present disclosure synthesized AFSNPs by mixing TEOS and APTES in different concentration proportions as shown in FIG. 4(A-C). As the concentration of APTES was increased (30, 50 and 70 mol %), the morphological characteristics revealed that the AFSNPs particle size increased to 224±8.4, 279±18.0 and 384±44.6 nm, respectively, and the surface appearance of the particles became rougher due to the non-hydrolysed and non-condensed of amine function (—NH₂). FIG. 4D shows the surface charge measured by the (ζ-potential. It can be seen that the AFSNPs in all conditions showed positive charges compared to the bare SNPs which were negatively charged due to the oxygen atoms on their surface. This confirmed that the surface could be modified to have amine functional groups by adding APTES in the system.^{18, 21} However, a similar charge in the range ±32 mV to ±39 mV could not confirm the presence number of amines because the number of particles in the solution was different.

The energy-dispersive spectroscopy (EDS) analysis was performed to determine the elemental composition and mapping images of the SNPs, and the mapping images were acquired using the K-line spectra of the elements. It was observed by the inventors that the compositions have an inhomogeneous distribution in the nanoparticle, where there exist the zones with high content of O and Si for both samples, whereas the zones with low content of N are shown in only FIG. 4B. The results are consistent with the elemental composition of the O, Si, and N having an atomic ratio close to (4.5:1:0) for SNPs and (3.1:1:0.3) for AFSNPs, respectively. This indicates that the elements are localized in the nanoparticle in random form. Therefore, this confirms the silica-based nanoparticles structure.

The Fourier transform infrared (FTIR) spectroscopy of SNPs and AFSNPs (conventional and one-step) shows peaks at 1049 cm⁻¹ to 1100 cm⁻¹ and around 795 cm⁻¹, which are attributed to stretching vibration of Si—O—Si bonds in the spine and free silanol group, respectively.¹⁰ The absorption peak around 939 cm⁻¹ to 945 cm⁻¹ is indicative of Si—OH vibration²⁷, compared with SNPs and AFSNPs (conventional), the AFSNPs (one-step) showed weaker absorbance than the SNPs. The reason is that part of the Si—OH has been replaced by the bending vibrations of —NH (primary) in APTES as a new band appeared at 694 cm⁻¹, while this peak did not appear in ASFNPs (conventional) because there was no crosslink between TEOS and APTES.

Moreover, the AFSNPs (conventional) showed broad peaks at 3260 cm⁻¹ and 1628 cm⁻¹ which should be attributed to —NH₂ stretching and bending, respectively.^{29, 30} It indicated that nanoparticle surface was successfully modified by comonomers of TEOS and APTES. It is noteworthy that the —NH₂ stretching and bending bands of ASFNPs (one-step) are shifted to lower wavenumbers by the hydrogen bonding³¹, which leads to high reactivity with molecules and specific interactions. In the comparison of the ASFNPs (one-step) spectrum at the different concentrations of added APTES, it was found that the intensity of 70 mol % APTES was higher than that of 50 mol % APTES at a peak around 694 cm⁻¹.

### Example 3

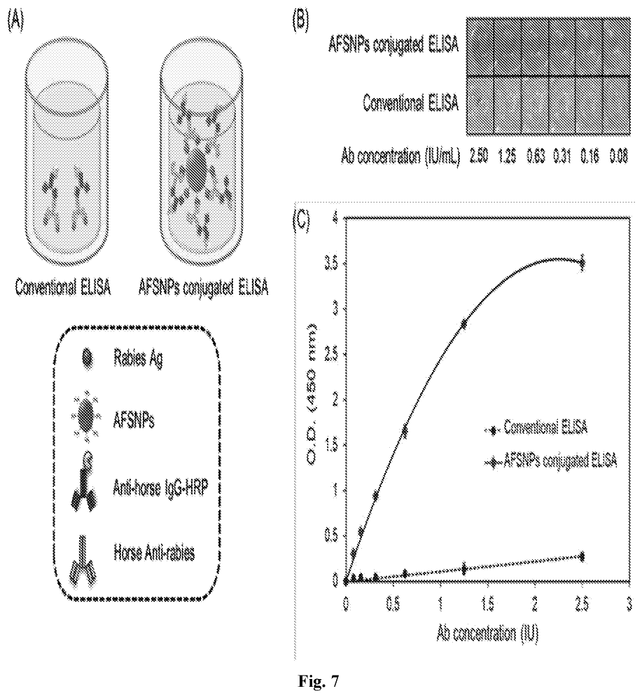

Cross-linking reactions were carried out for 30 min at room temperature in Eppendorf tube 1.5 mL with a mixture of itaconic acid, distilled water, EDC, and NHS. Then, AFSNPs and rabies virus antigen were added to the mixture at the same time. AFSNPs were incubated with rabies virus antigen (concentration of 0.4 μg, 0.2 μg, 0.1 μg, 0.05 μg, 0.025 μg, and 0.0125 μg, respectively) for 1 h at 25° C. The functionalized NPs were then centrifuged at 4000 rpm for 10 min and the precipitated NPs were resuspended in wash buffer three times to remove the excess antigen. The supernatant was collected for the antigen quantitation. As illustrated in FIG. 2, a newly developed crosslinker was used to bind between amine-terminated biomolecules and amine-terminated ASFNPs through covalent bonding. The efficiency of amine concentration, the stability of biomolecule immobilization as show in FIG. 6, and the performance of AFSNPs were examined through the ELISA technique. The signals in anti-rabies virus detection by ELISA compared between SNPs and AFSNPs synthesized in different concentrations of APTES. It was found that the concentration of APTES added to the particle synthesis system affected the particle morphology in terms of size and surface roughness. It also effects the amine content at the particle surface, which consequently affects the antigen conjugation. Since antigen is a protein that needs primary amines³³ for binding on the functionalized crosslinkers through using the itaconic coupling.

### Example 4

The 96-well ELISA plates (Immune; Nunc) were coated with antigen-immobilized AFSNPs (0.01 mg ml) at 4° C. for overnight. Then, they were washed with washing buffer three times and were blocked with blocking buffer for 6 mins. After the blocking buffer was removed, the diluted solution of antibody (100 μL) with desired concentration (2.50, 1.25, 0.63, 0.31, 0.16, and 0.08 IU/mL) was then added to each well and incubated for 1 hour at room temperature. Next, the well plate was washed with washing buffer for seven times, followed by addition of rabbit anti-horse IgG (whole molecule)-HRP 100 μL/well at optimized dilution of 1:3000) and incubated for 1 hour at room temperature. The well plate was washed with washing buffer for seven times again. Later, the TMB substrate was added to each well and incubated for 15 mins at room temperature. Finally, the reaction was stopped by adding stop solution and the absorbance of the product was measured at wavelength 450 and 570 nm using a microplate reader (Infinite® M Plex, Tecan Trading AG, Switzerland).

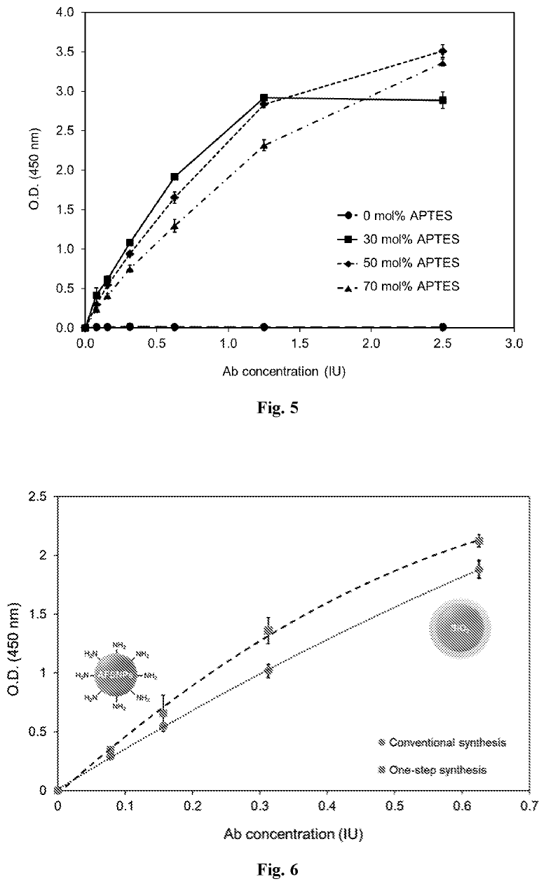

In FIG. 5, the SNPs with 0 mol % of APTES show no ELISA signal indicating that the antigen could not be immobilized on the surface of the SNPs, whereas the AFSNPs showed higher ELISA signal as the antigen concentration increased. At the beginning of the graph, despite having the lowest number of APTES 30 mol % exhibited the highest slope due to its smallest particle size and largest surface area for antibody immobilization.³⁴ However, due to the small amount of amine available on the surface used to immobilize the antibody, at the saturation point, there was insufficient antigens remained for immobilization of the antibody despite increased concentration of the antibody, thus the graph becomes plateau. At the concentrations of 50 mol % and 70 mol % APTES, the O.D. continued to increase with higher amount of antibody being captured due to the large number of amines available on the surface enhancing the immobilization efficiency. In view of that, inventors of the present disclosure believes that the one-step synthesis technique can be used to design and quantify antibody, antigen, peptides and the like on a surface of a testing platform incorporated with the synthesized and functionalized AFSNPs.

Furthermore, inventors of the present disclosure determined and compared the biomolecular binding capacity of AFSNPs by conventional and one-step synthesis as shown in FIGS. 1 and 2. It was found that the one-step synthesis method of AFSNPs resulted in higher ELISA signals for anti-rabies virus detection compared to the conventional method. Additionally, this method offers the advantage of using less APTES in the synthesis process. Although, in this study the similar size of nanoparticles was used, the ELISA signal from the one-step synthesized nanoparticles was still high due to the arrangement of the amine (—NH₂) structure in the particles that made it feasible to immobilize between the nanoparticles and the antigens.³⁵ The highly sensitive ELISA based on the ASFNPs as nanocarriers of anti-rabies antigen by using an itaconic acid crosslinker in an indirect ELISA is shown in FIG. 7. The indirect ELISA technique employs a two-step process for detection.³⁶ Particularly, a horse anti-rabies virus (primary antibody) specific to the rabies antigen binds to the AFSNPs then an anti-horse IgG-HRP (labelled secondary antibody) binds to the primary antibody for detection, as shown in FIG. 7A. The anti-rabies virus detection performance between conventional and AFSNPs-based ELISAs was also compared. It is expected that when AFSNPs are applied to ELISA, the amount of antigen immobilization and the ELISA signal can be increased. This is due to the assumption of increasing the surface area and functionality of the synthesized AFSNPs. In FIG. 7B, the developed reaction product colour of the applied AFSNPs to ELISA showed significantly deeper yellow than the conventional ELISA. The absorbance was measured at 450 nm, it was seen that the absorbance of AFSNPs conjugated ELISA showed a much higher signal than the conventional ELISA method as shown in FIG. 7C. The limit of detection (LOD) of the coupling AFSNPs is about 0.05 IU, which is ten times more sensitive than the LOD of conventional ELISA (0.5 IU). The proof showed their superiority in terms of detection sensitivity due to the efficiency of the functionalization of the ELISA through the bioreceptors. AFSNPs can offer increased surface areas, which leads to greater capacity for biomolecule binding.³⁷ Therefore, the AFSNPs can enhance the amplifying signal and the detection of the ELISA technique, which is useful in the early diagnosis of infection with very low concentrations of biomarkers.³⁸

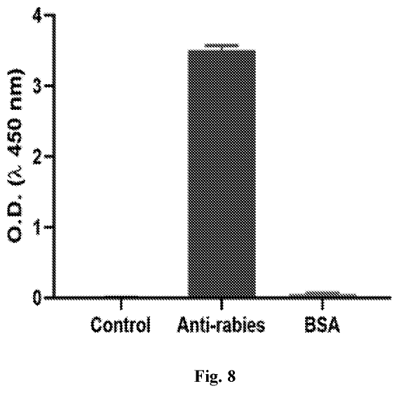

FIG. 8 shows the specificity of the proposed ELISA for anti-rabies virus detection. AFSNPs-rabies Ag was used to identify another sample, bovine serum albumin (BSA). The microplate was first coated with the AFSNPs-rabies Ag, BSA did not result in a colour change by demonstrating a same amount of O.D. value to the negative control. Additionally, a negative control could confirm non-immobilized rabies Ag if the itaconic acid crosslinker was not used in the system. However, the O.D. value of anti-rabies was increased by 60-fold when compared to BSA, indicating that the AFSNPs-based enhanced ELISA has high specificity for anti-rabies virus detection.

It is to be understood that the present disclosure may be embodied in other specific forms and is not limited to the sole embodiment described above. However, modification and equivalents of the disclosed concepts such as those which readily occur to one skilled in the art are intended to be included within the scope of the claims which are appended thereto

### REFERENCES

1. LaFleur, L.; Yager, P., Chapter II.5.13-Medical Biosensors. In Biomaterials Science (Third Edition), Ratner, B. D.; Hoffman, A. S.; Schoen, F. J.; Lemons, J. E., Eds. Academic Press: 2013; pp 996-1006.
2. Farka, Z.; Juřík, T.; Kovář, D.; Trnková, L.; Skládal, P., Nanoparticle-Based Immunochemical Biosensors and Assays: Recent Advances and Challenges. Chemical Reviews 2017, 117 (15), 9973-10042.
3. Martín-Gracia, B.; Martín-Barreiro, A.; Cuestas-Ayllón, C.; Grazú, V.; Line, A.; Llorente, A.; M. de la Fuente, J.; Moros, M., Nanoparticle-based biosensors for detection of extracellular vesicles in liquid biopsies. Journal of Materials Chemistry B 2020, 8 (31), 6710-6738.
4. Špringer, T.; Ermini, M. L.; Špačková, B.; Jabloňků, J.; Homola, J., Enhancing Sensitivity of Surface Plasmon Resonance Biosensors by Functionalized Gold Nanoparticles: Size Matters. Analytical Chemistry 2014, 86 (20), 10350-10356.
5. Altug, H.; Oh, S.-H.; Maier, S. A.; Homola, J., Advances and applications of nanophotonic biosensors. Nature Nanotechnology 2022, 17 (1), 5-16.
6. Gergeroglu, H.; Yildirim, S.; Ebeoglugil, M. F., Nano-carbons in biosensor applications: an overview of carbon nanotubes (CNTs) and fullerenes (C60). SN Applied Sciences 2020, 2 (4), 603.
7. Giner-Casares, J. J.; Henriksen-Lacey, M.; Coronado-Puchau, M.; Liz-Marzan, L. M., Inorganic nanoparticles for biomedicine: where materials scientists meet medical research. Materials Today 2016, 19 (1), 19-28.
8. Park, Y.; Jeong, S.; Kim, S., Medically translatable quantum dots for biosensing and imaging. Journal of Photochemistry and Photobiology C: Photochemistry Reviews 2017, 30, 51-70.
9. Yang, Y.; Zhang, M.; Song, H.; Yu, C., Silica-Based Nanoparticles for Biomedical Applications: From Nanocarriers to Biomodulators. Accounts of Chemical Research 2020, 53 (8), 1545-1556.
10. Baipaywad, P.; Kim, Y.; Wi, J.-S.; Paik, T.; Park, H., Size-controlled synthesis, characterization, and cytotoxicity study of monodisperse poly(dimethylsiloxane) nanoparticles. Journal of Industrial and Engineering Chemistry 2017, 53, 177-182.
11. Wagner, J.; Göβl, D.; Ustyanovska, N.; Xiong, M.; Hauser, D.; Zhuzhgova, O.; Hočevar, S.; Taskoparan, B.; Poller, L.; Datz, S.; Engelke, H.; Daali, Y.; Bein, T.; Bourquin, C., Mesoporous Silica Nanoparticles as pH-Responsive Carrier for the Immune-Activating Drug Resiquimod Enhance the Local Immune Response in Mice. ACS Nano 2021, 15 (3), 4450-4466.
12. Baipaywad, P.; Wi, J.-S.; Park, H.; Paik, T., Tailoring cubic and dodecagonal quasicrystalline mesophases of mesoporous organosilica nanoparticles and core/shell structure. Materials Science and Engineering: C 2019, 98, 666-674.
13. Lee, J. Y.; Kim, M. K.; Nguyen, T. L.; Kim, J., Hollow Mesoporous Silica Nanoparticles with Extra-Large Mesopores for Enhanced Cancer Vaccine. ACS Applied Materials & Interfaces 2020, 12 (31), 34658-34666.
14. Selvarajan, V.; Obuobi, S.; Ee, P. L. R., Silica Nanoparticles-A Versatile Tool for the Treatment of Bacterial Infections. Frontiers in chemistry 2020, 8, 602.
15. Janjua, T. I.; Cao, Y.; Yu, C.; Popat, A., Clinical translation of silica nanoparticles. Nature reviews. Materials 2021, 6 (12), 1072-1074.
16. Nayl, A. A.; Abd-Elhamid, A. I.; Aly, A. A.; Bräse, S., Recent progress in the applications of silica-based nanoparticles. RSC Advances 2022, 12 (22), 13706-13726.
17. de Oliveira, L. F.; Bouchmella, K.; Gonçalves, K. d. A.; Bettini, J.; Kobarg, J.; Cardoso, M. B., Functionalized Silica Nanoparticles As an Alternative Platform for Targeted Drug-Delivery of Water Insoluble Drugs. Langmuir 2016, 32 (13), 3217-3225.
18. Wang, Y.; Sun, Y.; Wang, J.; Yang, Y.; Li, Y.; Yuan, Y.; Liu, C., Charge-Reversal APTES-Modified Mesoporous Silica Nanoparticles with High Drug Loading and Release Controllability. ACS Applied Materials & Interfaces 2016, 8 (27), 17166-17175.
19. Miller, P. J.; Shantz, D. F., Covalently functionalized uniform amino-silica nanoparticles. Synthesis and validation of amine group accessibility and stability. Nanoscale Advances 2020, 2 (2), 860-868.
20. Widyasari, D. A.; Kristiani, A.; Randy, A.; Manurung, R. V.; Dewi, R. T.; Andreani, A. S.; Yuliarto, B.; Jenie, S. N. A., Optimized antibody immobilization on natural silica-based nanostructures for the selective detection of E. coli. RSC Advances 2022, 12 (33), 21582-21590.
21. Talreja, K.; Chauhan, I.; Ghosh, A.; Majumdar, A.; Butola, B. S., Functionalization of silica particles to tune the impact resistance of shear thickening fluid treated aramid fabrics. RSC Advances 2017, 7 (78), 49787-49794.
22. Samanta, D.; Sarkar, A., Immobilization of bio-macromolecules on self-assembled monolayers: methods and sensor applications. Chemical Society Reviews 2011, 40 (5), 2567-2592.
23. Masud, M. K.; Na, J.; Younus, M.; Hossain, M. S. A.; Bando, Y.; Shiddiky, M. J. A.; Yamauchi, Y., Superparamagnetic nanoarchitectures for disease-specific biomarker detection. Chemical Society Reviews 2019, 48 (24), 5717-5751.
24. Gloag, L.; Mehdipour, M.; Chen, D.; Tilley, R. D.; Gooding, J. J., Advances in the Application of Magnetic Nanoparticles for Sensing. 2019, 31 (48), 1904385.
25. Udomsom, S.; Mankong, U.; Paengnakorn, P.; Theera-Umpon, N., Novel Rapid Protein Coating Technique for Silicon Photonic Biosensor to Improve Surface Morphology and Increase Bioreceptor Density. 2021, 11 (5), 595.
26. Bootz, A.; Vogel, V.; Schubert, D.; Kreuter, J., Comparison of scanning electron microscopy, dynamic light scattering and analytical ultracentrifugation for the sizing of poly(butyl cyanoacrylate) nanoparticles. European Journal of Pharmaceutics and Biopharmaceutics 2004, 57 (2), 369-375.
27. Zhang, X.; Sun, Y.; Mao, Y.; Chen, K.; Cao, Z.; Qi, D., Controllable synthesis of raspberry-like PS—SiO2 nanocomposite particles via Pickering emulsion polymerization. RSC Advances 2018, 8 (7), 3910-3918.
28. Nakamoto, K., Infrared and Raman Spectra of Inorganic and Coordination Compounds, Part B: Applications in Coordination, Organometallic, and Bioinorganic Chemistry. Wiley: 2009.
29. Suteewong, T.; Sai, H.; Bradbury, M.; Estroff, L. A.; Gruner, S. M.; Wiesner, U., Synthesis and Formation Mechanism of Aminated Mesoporous Silica Nanoparticles. Chemistry of Materials 2012, 24 (20), 3895-3905.
30. Karnati, S. R.; Oldham, D.; Fini, E. H.; Zhang, L., Application of surface-modified silica nanoparticles with dual silane coupling agents in bitumen for performance enhancement. Construction and Building Materials 2020, 244, 118324.
31. Lin-Vien, D.; Colthup, N. B.; Fateley, W. G.; Grasselli, J. G., The Handbook of Infrared and Raman Characteristic Frequencies of Organic Molecules. Elsevier Science: 1991.
32. Koike, N.; Chaikittisilp, W.; Shimojima, A.; Okubo, T., Surfactant-free synthesis of hollow mesoporous organosilica nanoparticles with controllable particle sizes and diversified organic moieties. RSC Advances 2016, 6 (93), 90435-90445.
33. Saptarshi, S. R.; Duschl, A.; Lopata, A. L., Interaction of nanoparticles with proteins: relation to bio-reactivity of the nanoparticle. Journal of Nanobiotechnology 2013, 11 (1), 26.
34. Woeppel, K. M.; Zheng, X. S.; Cui, X. T., Enhancing surface immobilization of bioactive molecules via a silica nanoparticle-based coating. Journal of materials chemistry. B 2018, 6 (19), 3058-3067.
35. Gao, Y.; Zhou, Y.; Chandrawati, R., Metal and Metal Oxide Nanoparticles to Enhance the Performance of Enzyme-Linked Immunosorbent Assay (ELISA). ACS Applied Nano Materials 2020, 3 (1), 1-21.
36. Lin, A. V., Indirect ELISA. In ELISA: Methods and Protocols, Hnasko, R., Ed. Springer New York: New York, NY, 2015; pp 51-59.
37. Xing, Z. C.; Chang, Y.; Kang, I. K., Immobilization of biomolecules on the surface of inorganic nanoparticles for biomedical applications. Science and technology of advanced materials 2010, 11 (1), 014101.
38. Zhan, L.; Wu, W. B.; Yang, X. X.; Huang, C. Z., Gold nanoparticle-based enhanced ELISA for respiratory syncytial virus. New Journal of Chemistry 2014, 38 (7), 2935-2940.

1. A biomolecule-crosslinked compound of a chemical structure: 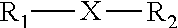
wherein X is a crosslinker of a formula of 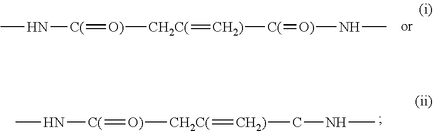
wherein R₁ is a silica nanoparticle and R₂ is a biomolecule incorporated with an amino-terminus capable of binding towards an amine functional group of the crosslinker.

2. The biomolecule-crosslinked compound of claim 1, wherein the crosslinker is produced by reacting itaconic acid, 1-Ethyl-3-(3-dimethyl aminopropyl) carbodiimide (EDC) and N-hydroxy succinimide (NHS) dispersed in an aqueous phase at a molar ratio of 15-35:60-90:30-60.

3. The biomolecule-crosslinked compound of claim 1, wherein the biomolecule is any of antigen, antibody, peptide, DNA or RNA.

4. The biomolecule-crosslinked compound of claim 1, wherein the silica nanoparticle comprises monomers and comonomers prepared in a molar ratio of 4.0:0.4 to 1.3:3.0.

5. The biomolecule-crosslinked compound of claim 4, wherein the monomers are Tetrapropyl orthosilicate (TPOS), Tetraethyl orthosilicate (TEOS), Tetramethyl orthosilicate (TMOS), Tetra-isopropyl orthosilicate or Butyl orthosilicate.

6. The biomolecule-crosslinked compound of claim 4, wherein the comonomers are selected from (3-Aminopropyl)-triethoxysilane), (3-Aminopropyl)-trimethoxysilane), (3-Aminopropyl-methyl-diethoxysilane), (3-(Dimethoxymethylsilyl) propylamine), (3-Aminopropylsilanetriol), (N-(2-Aminoethyl) (3-aminopropyl)methyldimethoxysilane) or N-(2-Aminoethyl) (3-aminopropyl) trimethoxysilane).

7. A method of producing a biomolecule-crosslinked compound comprising the steps of:
reacting a plurality of monomers and a plurality of comonomers in the presence of ammonia within a first solution to generate a compound;
retrieving the produced compound from the first solution for cleaning;
subjecting the cleaned compound to react with a second solution to join a crosslinker onto the compound through a first amine functional group of the crosslinker; and
adding a plurality of biomolecules to the second solution to bind the biomolecules towards the compound through a second amine functional group of the crosslinker to produce the biomolecule-crosslinked compound,
wherein the monomers are Tetrapropyl orthosilicate (TPOS), Tetraethyl orthosilicate (TEOS), Tetramethyl orthosilicate (TMOS), Tetra-isopropyl orthosilicate or Butyl orthosilicate, wherein the comonomers are selected from (3-Aminopropyl)-triethoxysilane), (3-Aminopropyl)-trimethoxysilane), (3-Aminopropyl-methyl-diethoxysilane), (3-(Dimethoxymethylsilyl) propylamine), (3-Aminopropylsilanetriol), (N-(2-Aminoethyl) (3-aminopropyl)methyldimethoxysilane) or N-(2-Aminoethyl) (3-aminopropyl) trimethoxysilane).

8. The method of claim 7, wherein the monomers and the comonomers are in a molar ratio of 4.0:0.4 to 1.3:3.0.

9. The method of claim 7, wherein the reacting step is performed at a temperature range of 20° C. to 40° C.

10. The method of claim 7, wherein the first solution comprises ammonium hydroxide dissolved in a mixture of distilled water and ethanol in a volume ratio of 1-10:20-30.

11. The method of claim 7, wherein the crosslinker is itaconic acid having both ends incorporated with an amine functional group respectively.

12. The method of claim 7, wherein the second solution comprises itaconic acid, 1-Ethyl-3-(3-dimethyl aminopropyl) carbodiimide (EDC) and N-hydroxy succinimide (NHS) dispersed in an aqueous phase at a molar ratio of 15-35:60-90:30-60.

13. The method of claim 7, wherein the biomolecule is any of antigen, antibody, peptide, DNA or RNA incorporated with an amino-terminus capable of binding towards the second amine functional group of the crosslinker.

14. A method of producing amine-functionalized silica nanoparticles (AFSNPs) comprising:
reacting a plurality of monomers and a plurality of comonomers in the presence of ammonia within a first solution to generate the AFSNPs; and
retrieving the produced AFSNPs from the first solution for cleaning,
wherein the monomers are Tetrapropyl orthosilicate (TPOS), Tetraethyl orthosilicate (TEOS), Tetramethyl orthosilicate (TMOS), Tetra-isopropyl orthosilicate or Butyl orthosilicate, and the comonomers are selected from (3-Aminopropyl)-triethoxysilane), (3-Aminopropyl)-trimethoxysilane), (3-Aminopropyl-methyl-diethoxysilane), (3-(Dimethoxymethylsilyl) propylamine), (3-Aminopropylsilanetriol), (N-(2-Aminoethyl) (3-aminopropyl)methyldimethoxysilane) or N-(2-Aminoethyl) (3-aminopropyl) trimethoxysilane).

15. The method of claim 14 further comprising subjecting the cleaned AFSNPs to react with a second solution to join a crosslinker onto the compound through a first amine functional group of the crosslinker, wherein the crosslinker is of a formula of: [chemical structure]
